(19) United States
(12) Patent Application Publication
(10) Pub. No.: US 2026/0198800 A1
(43) Pub. Date: Jul. 16, 2026

(54) Smart Footwear, Insoles or Other Wearables with Electronically Read Sensing Membrane and Self-Identification of Left/Right Status

(71) Applicant: 0315636 B.C. Ltd. (DBA Avriel International), Winnipeg (CA)

(72) Inventors: Bruce Griffith, Winnipeg (CA); Kevin Roorda, Winnipeg (CA); Riley Cornelius, Winnipeg (CA)

(21) Appl. No.: 19/428,770

(22) Filed: Dec. 22, 2025

Related U.S. Application Data
(60) Provisional application No. 63/741,582, filed on Jan. 3, 2025.

(51) Int. Cl. A61B 5/103; A61B 5/00

(52) U.S. Cl. CPC A61B 5/1036; A61B 5/0082; A61B 5/6807

(57) ABSTRACT

Wearable smart devices are disclosed that incorporate a unique means of detecting or measuring a substance of interest, for example to detect ammonia in the secreted foot sweat of a diabetic via incorporation of the sensing arrangement in a footwear article or insole. The setup employs the combination of a colour-changing sensing membrane responsive to the substance of interest, an optical sensor appropriately positioned to capture the membrane within its field of view, and an illumination means for illuminating the membrane to enable optical imaging thereof, from which a controller can then detect the colour of the membrane and determine the presence or measurement of the substance of interest. A left/right self-identification circuit is included in each wearable smart device to enable a cooperative pair of such devices, worn on different bilateral appendages, to self-identify their left/right designation to the external device that receives the outputted signals from the wearables.

### CROSS-REFERENCE TO RELATED APPLICATIONS

### This application claims benefit under 35 U.S.C. 119(e) of U.S. Provisional Application No. 63/741,582, filed January 3, 2025, the entirety of which is incorporated herein by reference

### FIELD OF THE INVENTION

The present invention relates generally to sensor-equipped wearables, and particularly use of such technology for monitoring health of diabetics, or other populations similarly susceptible to health complications.

### BACKGROUND

Diabetes is a chronic hormonal disorder that can impact the body's ability to produce and utilize insulin, resulting in inadequate regulation of blood sugar levels. In Canada, the prevalence of diabetes was approximately 2.3 million in 2017, with an anticipated addition of about 2.16 million new cases by 2022. The projected influx of new diabetes cases is expected to contribute to approximately $15.36 billion in Canadian healthcare costs, with a significant portion allocated to acute hospitalizations and physician services. Diabetic foot disorders are deemed medical emergencies and can progress to a severity necessitating amputation, representing the second most feared comorbidity of diabetes after blindness. Addressing the challenges posed by diabetic foot disorders is crucial not only for individual health but also for managing the substantial economic burden on healthcare systems. While single metrics such as temperature, plantar pressure/force, gait changes, and blood flow have individually demonstrated indications of ulceration, none of them can be solely relied upon as wholly reliable predictors of ulceration. Recognizing this limitation, the development of a wearable smart sensing system becomes crucial. Such a system would ideally have the capability to integrate measurements from multiple factors simultaneously, offering a multifactorial pathway for predicting tissue failure. The advantage lies in the comprehensive analysis provided by combining various metrics, enhancing the system's ability to predict and potentially prevent ulceration more effectively than relying on individual metrics alone.

While there are numerous prior patents for various designs of “smart insoles” with force sensor arrays for detecting and measuring problematic pressure points on the soles of a user’s feet, there has also been a realization that there could be valuable in measuring other conditions inside a user’s footwear that could also be useful in the context of monitoring foot health. Published US Patent Application 2024/0277106 in the name of Tao Treasures LLC (DBA NonBioFAB), for example, discloses a smart insole that sees supplementation of its array of force sensors with a gas sensory array for detecting volatile organic compounds (VOCs), of which acetone, ammonia and nitrogen dioxide are a few named examples of gases that may emanate from the skin and be useful indicators of foot health. While different classifications of potentially useable gas sensing technologies are briefly mentioned (electrochemical, chemiresistors, metal oxides, infrared, or optical sensors), the drawings are purely schematic, and no detailed working examples are given, demonstrating an unfulfilled need on this front.

Another area with room for improvement is minimization of the risk of possible user error in setup of the smart insoles, which would be typically be used in combination with a software application running on a user’s smartphone, to which the respective insoles of the user’s left and right footwear articles would be paired. It would be ideal to eliminate, or reduce as much as possible, the chance of user error in the wireless pairing of the smart insoles to the smartphone to ensure proper assignment of the left and right insoles to the left and right measurement, logging and display functionalities of the software application, to ensure that the left foot and right foot measurements are being properly mapped as such. To this end, Published US Patent Application 2024/0090620 in the name of LAAF, Inc. discloses that its left and right insoles wirelessly advertise themselves with unique Bluetooth names that respectively incorporate left and right designations within their names so that, during wireless electronic pairing of the smart insoles to the smartphone, the left and right insoles are properly mapped to the software application. However, no detailed implementation is given in terms of how each insole identifies its left or right footed designation.

In the meantime, Applicant has been working on development of smart insole product to address the unfulfilled market need, and in doing so, has derived unique solutions addressing the above shortcomings of the prior art and extendable to other wearable smart devices, the details of which will be understood from the detailed description given further below, with reference to the accompanying drawings.

### SUMMARY OF THE INVENTION

According to a first aspect of the invention, there is provided a wearable smart device with sensing capability, said wearable smart device comprising:

a wearable body wearable in adjacency to a body part of a user; and

in physical accompaniment to said wearable body for combined wearing therewith:

a colour-changing sensing membrane positioned at a location exposable to a substance of interest during worn use of the wearable device by said user;

an optical sensor positioned to encompass at least a portion of the colour-changing sensing membrane within a field of view of said optical sensor; and

a light source positioned and operable to illuminate at least said portion of the colour-changing sensing membrane; and

an electronic controller operatively coupled to said optical sensor and light source, and configured to trigger:

execution of measurements comprising:

activation of the light source to cause illumination of at least said portion of the colour-changing sensing membrane; and

during said illumination, obtaining an output signal from the optical sensor that comprises, at least, colour data representative of a current colour of the colour-changing sensing membrane.

According to a second aspect of the invention, there is provided a sensing method comprising:

hosting, by a wearable smart device, of a sensing setup comprising:

a colour-changing sensing membrane positioned at a location exposable to a substance of interest during worn use of the wearable smart device;

an optical sensor positioned to encompass at least a portion of the colour-changing sensing membrane within a field of view of said optical sensor; and

a light source positioned and operable to illuminate at least said portion of the colour-changing sensing membrane; and

an electronic controller operatively coupled to said optical sensor and light source; and

by operation of said electronic controller, executing measurements comprising:

activation of the light source to cause illumination of at least said portion of the colour-changing sensing membrane; and

during said illumination, obtaining an output signal from the optical sensor that comprises, at least, colour data representative of a current colour the colour-changing sensing membrane.

According to a third aspect of the invention, there is provided a wearable smart device with sensing capability, said wearable smart device comprising:

a wearable body wearable on one of two bilateral appendages of a user;

in physical accompaniment to said wearable body, at least one sensor operable to take measurements of one or more measurable conditions during worn use of the wearable smart device by said user;

an electronic controller operatively coupled to said at least one sensor to take said measurements therefrom; and

a transmitter connected to said electronic controller and operable to transmit reporting signals, to an external device, based at least partially on said measurements taken from the at least one sensor;

wherein said electronic controller is configured to self-detect whether the wearable smart device is adapted for a left or right one of the bilateral appendages based on an automated reading, by the electronic controller, of a left/right self-identification circuit coupled to said electronic controller.

According to a fourth aspect of the invention, there is provided a method of setting up a pair of sensor-equipped wearable smart devices, intended for wearing on a different two bilateral appendages of a user, for cooperation with an external receiving device for receiving reporting signals from said sensor-equipped wearable smart devices, said method comprising:

(a) by operation of a respective controller residing with each of said wearable smart devices, self-identifying each of said wearable smart devices as either a left appendage device or right appendage device by automated reading of a left/right self-identification circuit coupled to said respective controller; and

(b) in electronically pairing respective transmitters said pair of wearable smart devices to the external receiving device, using a self-identified left-appendaged status of one of said wearable smart devices to map the reporting signals therefrom as left-appendage readings and likewise using a self-identified right-appendaged status of one of said footwear to map the reporting signals therefrom as right-appendage readings.

### BRIEF DESCRIPTION OF THE DRAWINGS

Preferred embodiments of the invention will now be described in conjunction with the accompanying drawings in which:

FIG. 1 is a top front perspective view of a smart footwear insole of the present invention, shown in a left footed configuration, and that in use is paired with another insole having a right footed configuration of mirror imaged relation to the illustrated insole.

FIG. 2 is an isolated top front perspective view of an upper body layer of the insole of FIG. 1.

FIG. 3 is an isolated bottom front perspective view of a lower body layer of the insole of FIG. 1.

FIG. 4A is a top plan view of a circuit board assembly and associated power componentry of the smart footwear insole of FIG. 1, for sandwiched installation between the upper and lower body layers of FIGS. 2 and 3.

FIG. 4B is another top plan of the circuit board assembly and power componentry of FIG. 4A, with sensors installed at sensor pads of a flexible PCB of the assembly, and after potting of the sensor pads and a main PCB of the assembly.

FIG. 5 is a top plan view of the smart footwear insole of FIG. 1 prior to installation of a colour-changing sensing membrane thereon.

FIG. 6 is a schematic and partial cross-sectional view of the insole of FIG. 5 as taken long line 6 – 6 thereof after installation of the colour-changing sensing membrane thereon.

FIG. 7 is a schematic diagram of an ammonia detector of the smart footwear insole of FIG. 1 that performs optical colour measurement of the colour-changing sensing membrane.

FIG. 8 schematically illustrates left/right self-identification circuitry employed in a left and right pair of smart insoles of the type illustrated in FIG. 1.

FIG. 9 is a screenshot of a sensor data display screen in a graphical user interface of a software application running on a smartphone paired with a set of left and right smart insoles of the type illustrated in FIG. 1.

FIG. 10 is a screenshot of a setup screen in the graphical user interface of the software application.

FIG. 11A is a top plan view of the main PCB of the circuit board assembly of FIG. 4A, which is configured for use in a right-footed insole.

FIG. 11B is a top plan view of a similar main PCB of a circuit board assembly configured for use in a left-footed insole.

### DETAILED DESCRIPTION

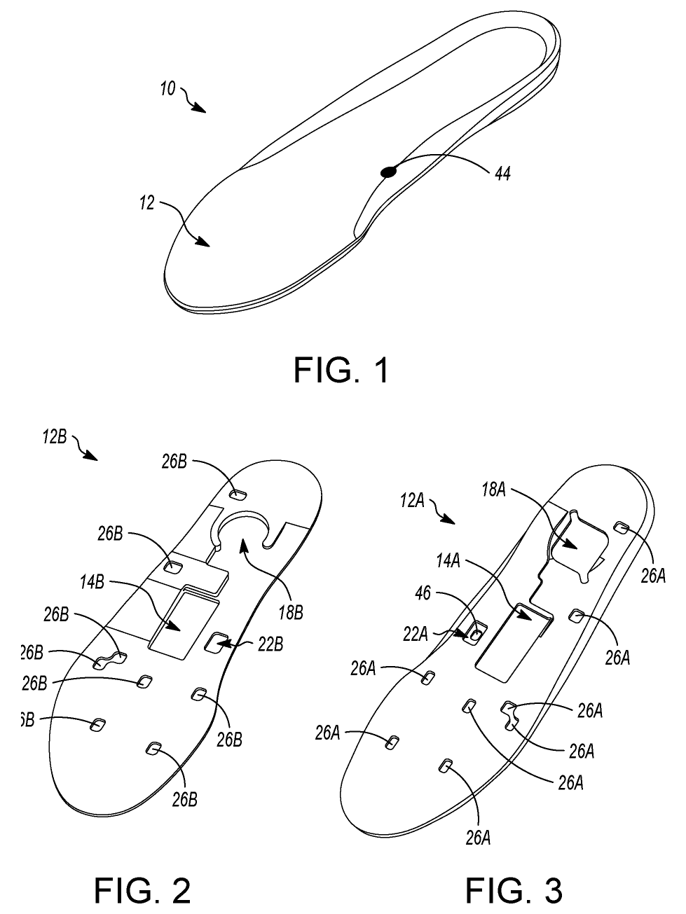

FIG. 1 illustrates, in a fully assembled form thereof, a sensor-equipped footwear insole, or smart insole, 10 of the present invention that is equipped with a suite of sensors for electronically measuring, and reporting taken measurements of, different conditions experienced during wearing of a footwear article in which the insole has been installed. The measurable conditions may include force exerted on the insole by the user’s foot at various force-monitored locations distributed throughout the footprint area of the insole, temperature experienced at a matching or different set of temperature-monitored locations likewise distributed throughout the footprint area of the insole, and the presence of one or more particular substances in the internal environment of the footwear, particularly one or more substances that are usable as an indicator of foot health, of which ammonia is the particular example used in the context of the detailed embodiments set forth herein below, though the inventive apparatus and methodology disclosed herein for ammonia detection purposes may be used or modified for similar detection of other substances, among which there may be included any one or more of the subject VOC’s that were targeted as substances of interest in the prior art cited above.

A substantial volume of the insole 10 is embodied by an insole body 12 typically composed of a foam (e.g. polyurethane foam) or other compressible substance given that, in addition to the sensing capability imparted by the onboard electronics of the smart insole 10, the smart insole also serves the comfort-imparting functionality of a conventional sensorless insole, to provide comfortable padding atop the hard sole of the footwear during wearing thereof by the user. In order to house the electronic componentry that imparts the sensing capability of the insole 10, the insole body 12 is composed of multiple layers, by which electronic componentry can be housed in a substantially enclosed/embedded fashion within the overall volume of the insole body 12 in sandwiched relationship between any two or more adjacently stacked layers. In the illustrated embodiment, the insole body 12 is composed of only two such stacked layers, a top layer 12A and a bottom layer 12B, and the full suite of electronic componentry is housed between those two layers 12A, 12B.

The topside of the bottom layer 12B and/or the underside of the top layer 12A, and more particularly both thereof in the illustrated embodiment, have recessed cavities therein at different locations distributed throughout the footprint area of the insole to host respective members of the various electronic componentry. In the illustrated embodiments, these recessed cavities include top and bottom PCB cavities 14A, 14B of matching location on the top and bottom layers 12A, 12B for cooperative holding therein of a main printed circuit board (PCB) 16 of the electronic componentry, top and bottom power component cavities 18A, 18B of matching location on the top and bottom layers 12A, 12B for cooperative holding therein of one or more power components 20 of the electronic componentry, top and bottom optical sensor cavities 22A, 22B of matching location on the top and bottom layers 12A, 12B for cooperative holding therein of an optical sensor setup 24 of the electronic componentry, and a plurality of paired top and bottom force/temperature sensor cavities 26A, 26B, of the which the two cavities of each pair are of matching location on the top and bottom layers 12A, 12B for cooperative holding therein of a respective force/temperature sensing node 28 of a force/temperature sensing array of the smart insole 10. Except where stated otherwise, the expression force/temperature is used herein to mean force and/or temperature, and so a force/temperature sensing node 28 may comprise a force sensor, a temperature sensor, or the combination thereof.

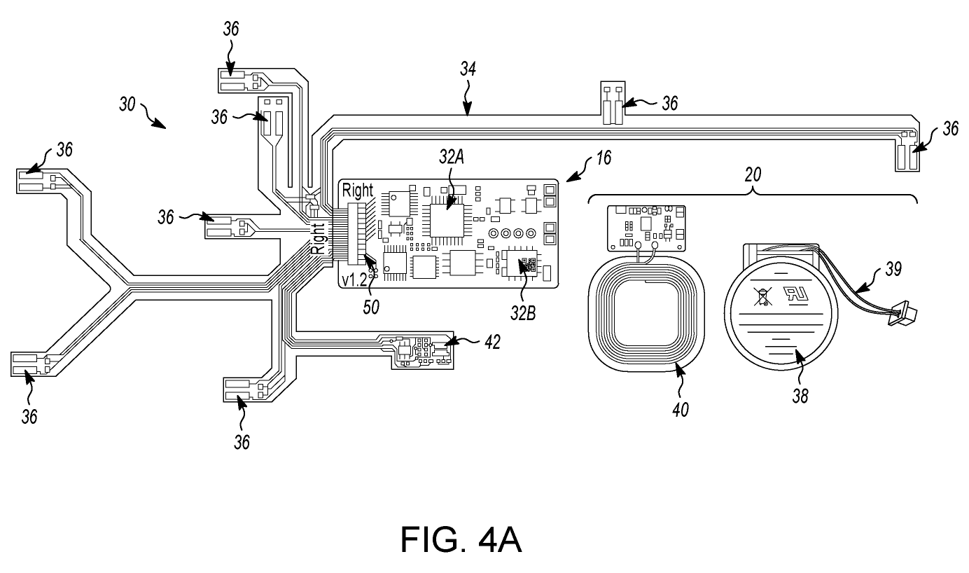

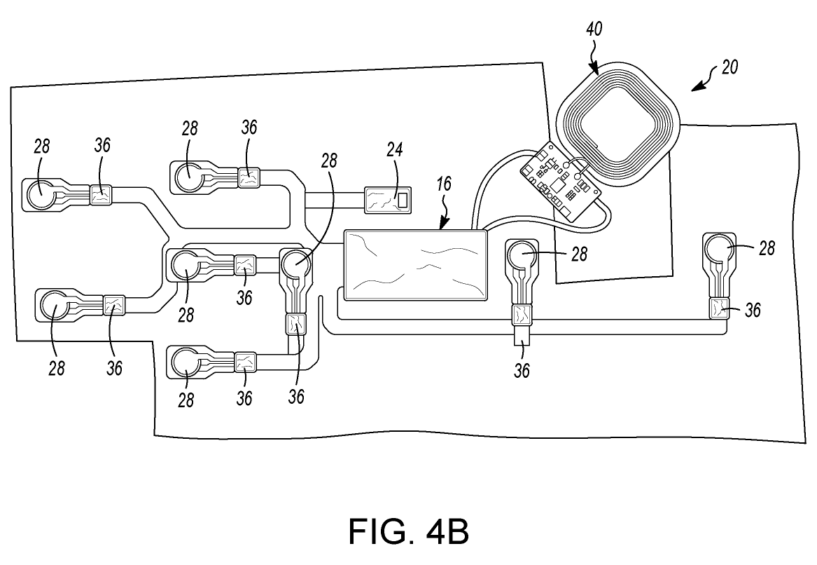

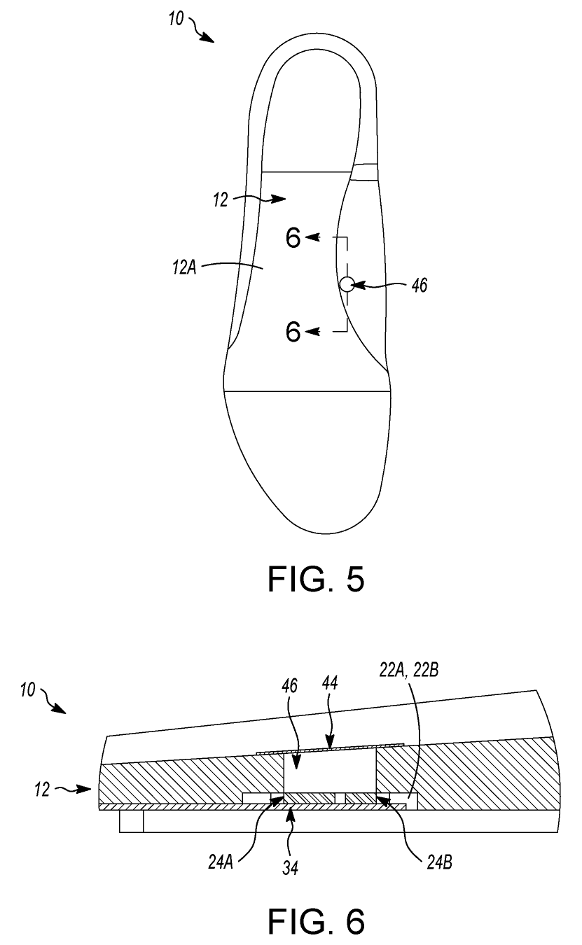

FIG. 4A illustrates a circuit board assembly 30 of the smart insole 10, in isolated relationship from the layered sole body 12 in which it is installed during factory production of the smart insole. The circuit board assembly 30 features the aforementioned PCB 16 that hosts a controller (typically embodied in one or more microcontrollers 32A, 32B), a transmitter for wireless communication of reporting signals to an external receiving device (typically a smartphone), flash memory for storing measurement data derived from readings of the various sensors for transmission of such measurement data within said reporting signals, a battery management system, voltage regulation circuitry and any necessary sensor conditioning components. In a prototype demonstrative of one preferred embodiment, the controller comprises a primary microcontroller 32A (e.g. STM32F103C8T6) responsible for running firmware responsible for a bulk of the smart insole’s functionality, and a secondary microcontroller 32B (e.g. RN4871U) serving as a controller of the wireless transmitter, typically embodied as a Bluetooth transmitter, and leaving all operations other than wireless communication to the primary microcontroller.

In addition, the circuit board assembly 30 features a flexible printed circuit board (FPCB) 34 for hosting the various sensors and having thereon the necessary circuit traces for conductively connecting those sensors to the main PCB 16 to enable signal communication between those sensors and the controller 32A to enable taking of sensor measurements thereby. The FPCB features a plurality (eight in the illustrated example) of force/temperature sensor pads 36, mostly, if not entirety, situated at the ends of respective branches of the FPCB 34, for respectively hosting an equal quantity of force/temperature sensing nodes 28 at respective locations branched out from the main PCB 16 in matched distribution to the locations of the force/temperature sensor cavities 26A, 26B in the top and bottom layers 12A, 12B of the insole body 12. The power component cavities 18A, 18B may respectively, or collectively, store therein both a rechargeable battery 38, coupled to the main PCB 16 in the nearby PCB cavity via wiring a harness 39, and a wireless charging coil 40 for charging that battery 38 in wireless fashion from outside the sole body 12 by a wireless charger (not shown).

Smart insoles with wireless battery charging and with force sensors distributed at spaced locations throughout the insole for measurement of force exerted thereon by the foot of the user at such locations, and for transmission of the measured force data onward to a smartphone running a compatible software application thereon are known in the art, and so further descriptive detail of these particular functionalities of the smart insole 10 and the associated componentry is omitted in the interest of brevity, and primary focus instead given to other details of the present embodiment that are believed novel and inventive over such prior work. What brief description is made herein of the force/temperature sensors, charging coil and associated componentry and functionality is made primarily for the purpose of setting one non-limiting example of an operating context for the novel and inventive subject matter disclosed and claimed herein, which prove particularly useful in a smart insoles that includes such force and/or temperature measuring means and wireless charging capability, but may also be included in smart insoles whose “smart” electronically-enabled functionality does necessarily include force and/or temperature measurement, and regardless of whether charging of the insole’s onboard battery is via wireless charging or via a selectively connectable charging cable. The smartphone or other external receiving device will typically have one or more processors, computer readable memory coupled thereto in which a software application is stored as executable statements and instructions for execution by the one or more processors to perform any and all tasks ascribed herein to such software, and a display for displaying a graphical user interface (GUI) of that software application.

The FPCB 34 also includes an optical sensor setup pad 42 thereon at the respective end of one branch of the FPCB 34 for the purpose of hosting the optical sensor setup 24 in a manner conductively and communicatively linked to the controller 32A of the main PCB 16 via traces of the FPCB 34, though in other embodiments, the optical sensor setup 24 could instead be hosted on the main PCB 16, or on a smaller dedicated PCB wired to the main PCB 16 to make use of the same controller 32A as the other sensors (force/temperature nodes 28). The optical sensor setup 24 installed atop the optical sensor setup pad 42 features a closely neighboured pairing of an optical sensor 24A and an accompanying light source 24B, typically a white light-emitting diode (LED), the combined purpose of which is to enable measurement of the current colour (at any instance of time) of a colour-changing sensing membrane 44 that is responsive in colour-changing fashion to the presence of a targeted substance of interest, such as ammonia. Colour-change ammonia sensing membranes are a known type of product already commercially available for other substance-detection applications, in view of which no detailed disclosure of the composition and manufacture of such membrane need be made herein to enable practice of the present invention, which detail is therefore omitted in the interest of brevity.

In the illustrated embodiment, the top optical sensor cavity 22A in the underside of the top layer 12A of the insole body 12 is accompanied by a communicative opening 46 that penetrates through to the topside of the top layer 12A of the insole body 12, which also denotes the topside of the overall insole body 12, atop which the sole of the user’s foot stands in regularly worn use of the footwear in which the smart insole 12 is installed. In the example shown in FIGS. 1 and 6, the colour-changing sensing membrane 44 is installed on the topside of the insole body 12 in overlying relationship to the communicative opening 46. The optical sensor 24A and accompanying light source 24B reside beneath the top layer 12A of the insole body 12, within the optical sensor cavity 22A recessed in the underside thereof, and in alignment with the communicative opening 46. This way, the field of view (FOV) of the optical sensor 24A, through the communicative opening 46 thereabove, encompasses at least a partial area of the colour-changing sensing membrane 44 that is fixed atop the insole body 12 in spanning relationship over the communicative opening 46. Similarly, the light cast by the light source 24B, when activated, and owing to exposure of the light source 24B and the colour-changing sensing membrane 44 to one another through the communicative opening 46, illuminates at least the same portion of the colour-changing sensing membrane 44 that is optically visible to the optical sensor 24A.

The location of the optical sensor cavity 22A and the optical sensor setup 24 installed thereat is preferably located with an arch-underlying mid-region of the insole body 12 that resides beneath the arch of the wearer’s foot in the worn state of the footwear, as opposed to residing beneath the heel or ball of the foot. Such arch-based placement of the optical sensor setup 24 has the benefit of reduced impact under the body weight of the wearer, and also exposure to greater airflow compared to those areas, the latter of which may be most significant particularly when the targeted substance of interest is ammonia or another gaseous substance, though similar setup of an optical sensor setup may be used to monitor for liquid substances of interest in other embodiments. The optical sensor setup may reside within a central 40% of the insole’s overall length in some embodiments, within a central 30% of the insole’s overall length in some embodiments, and within a central 20% of the insole’s overall length in some embodiments.

In the illustrated example representative of prototyped insoles, the placement of the optical sensor setup 24, in addition to residing within an arch-underlying mid-region of the insole, is medially offset from a widthwise center of the insole, and resides closer to a medial inside edge of the insole than to a lateral outside edge thereof. The location of the optical sensor setup, for example using a central axis of the optical sensor cavity 22A or communicative opening 46 as a reference point to define such location, may reside within a medial 40% fraction of the insole’s width (for example, meaning a localized reference width of the insole as measured at the location of the optical sensor setup) in some embodiments, within a medial 35% fraction of this localized reference width of the insole in some embodiments, and within a medial 30% fraction of this localized reference width of the insole in some embodiments.

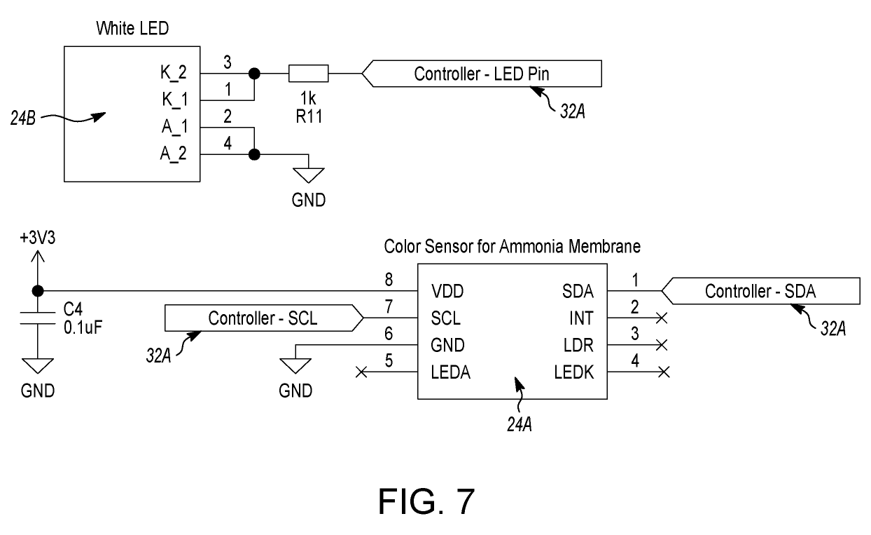

The optical sensor 24A and light source 24B are operatively connected to the controller 32A on the main PCB 16, via the traces of the FPCB 34, as schematically illustrated in FIG. 7, so that the light source 24B is selectively activatable by the controller 32A at every instance in which the controller 32A calls for a reading from the optical sensor 24A, which call therefore includes, in sequence, activation of the light source to cause illumination of the colour-changing sensing membrane, reading of a signal from the optical sensor in which there is embodied colour data representative of the instantaneous colour currently possessed by the colour-changing sensing membrane at that moment of optical capture, and deactivation of the light source in the interest of power conservation, given that illumination is only necessary when triggering a reading of the sensing membrane colour by the optical sensor 24A.

As part of the algorithm, the colour data from the optical sensor may be transformed from one colour data format to another, for example from RGB (red / green / blue) to HSL (hue / saturation / lightness/luminance). Since the colour-changing sensing membrane 44 changes colour in a predictable and measurable way when exposed to the target substance (in this case, ammonia gas), the periodic measurement of the hue of the colour membrane can track changes in ammonia exposure with respect to time. The controller timestamps and stores each hue measurement in flash memory, and transmits reporting signals, including at least those timestamped hue measurements, via the transmitter, to the smartphone or other external device, on which a running software application may do any number of useful things with the received reporting data, including display of all or a subset thereof in a user interface, triggering of alarms or notifications of potential problems signified by the reporting data, uploading of all or a subset of the reporting data to a remote (e.g. cloud) database for logging and/or immediate or future analysis.

Prototypes of the invention were produced using commercially available colour-change ammonia gas sensor membranes from Pacific Sentry, one designed to detect ammonia levels of 1 ppm to 5+ ppm, and the other to detect ammonia levels of 1 ppm to 50+ppm. These sensor membranes change from canary yellow to a deep blue colour in the presence of ammonia and revert to their baseline canary yellow when ammonia is no longer present. In some embodiments, the software on the smartphone or other external receiving device may be configured to trigger an alarm or notification of a potential foot health problem if a quantity of positive ammonia detections exceeds a predetermined threshold, which optional implementation is based on the non-limiting theory that a single report of higher than normal ammonia concentration may not denote a health complication requiring medical attention, whereas ongoing elevated ammonia readings over a period of time is a more significant indication that medical assessment or other intervention may be warranted.

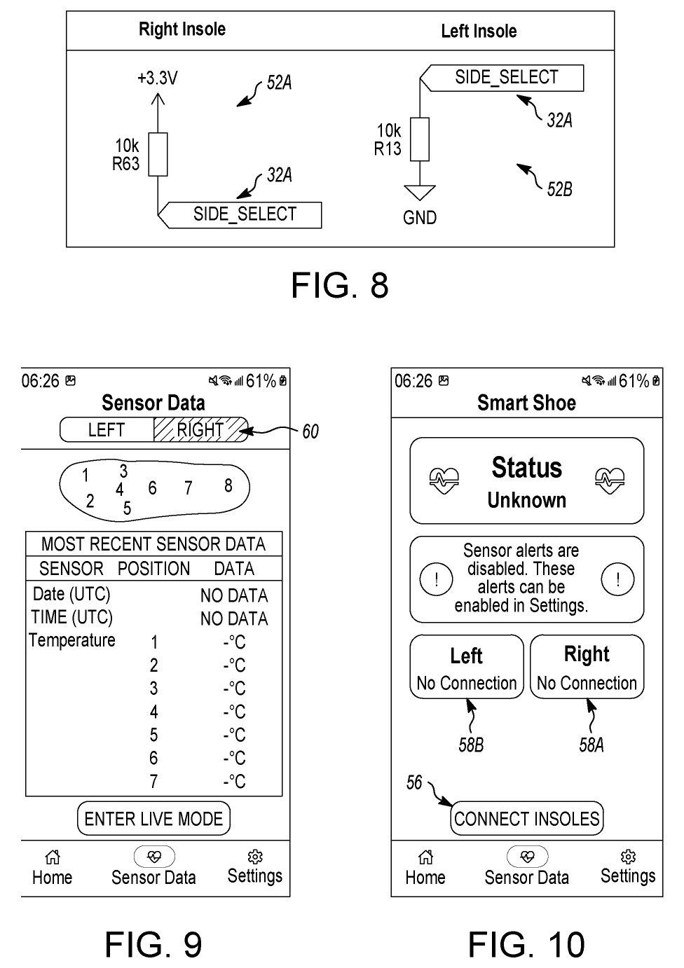

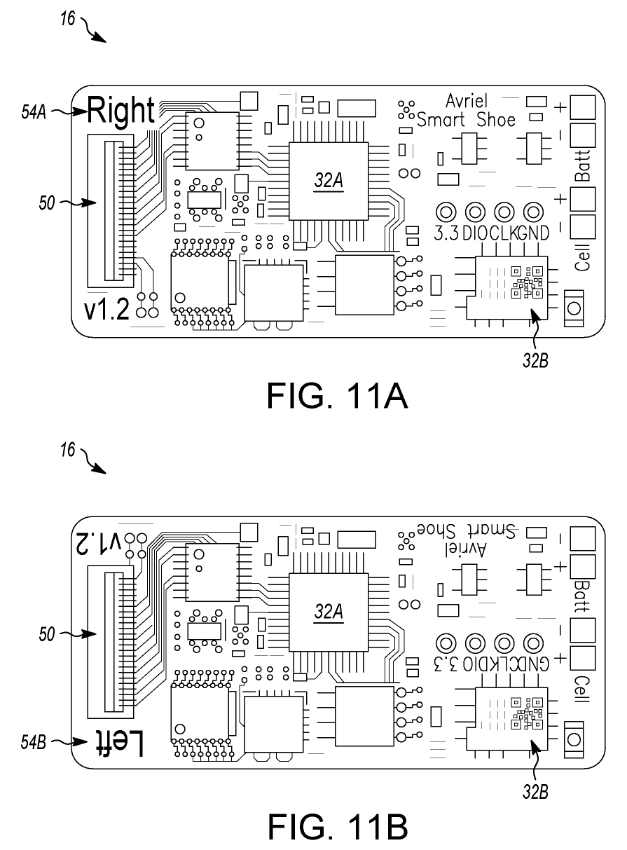

For manufacturing and part count efficiency, any given size of insole may use the exact same FPCB 34 for both the left-footed and right-footed version of the insole, in which case the FPCB is characterized by inclusion soldering pads on both sides of the FPCB at each sensor pad 36, 42. This way, the two respective FPCBs 34 of the two circuit board assemblies 30 for a left and right pair of insoles differ only in reversal of the orientations in which the FPCBs are assembled with the sensors and the PCBs 16 of those circuit board assemblies 30. The two PCBs may also be of substantially the same design and manufacture, with a minimal number of minor modifications between the two according to the left-footed or right-footed status of the insole to which they respectively belong. In the illustrated example, there are three such minor modifications: one being a differently mapped set of circuit traces to the controller 32A from the connection terminal 50 at which plugged connection of the FPCB 34 to the PCB 16 takes place, given the aforementioned difference in mirrored orientation of the FPCBs 34 of the left and right insoles; another being selection between two different configurations of a self-identification circuit 52A, 52B (see FIG. 8) on the PCB 16 by which the controller 32A is able to self-detect whether it belongs to a left-footed or right-footed insole; and yet another being marking of the PCB with an installer-readable label 54A, 54B (again see FIGS. 11A & 11B) indicative of whether the controller is configured for installation in a left-footed or right-footed insole during factory production thereof.

The first and third of these minor modifications between the left and right versions of the PCB 16 are self-evident, and require no further description. The self-identification circuit 52A, 52B is shown in its two possible configurations in FIG. 8. A same singular input pin on the controller 32A of the PCB 16 in each of the left-footed and right-footed circuit board assembly is used for the self-identification of the insole as either left-footed or right-footed, which pin is schematically denoted as “side-select” pin in FIG. 8. In the illustrated right-footed configuration of the self-identification circuit 52A, this side-select pin of the controller 32A is connected to an operating voltage of the PCB 16 (e.g. 3.3V), while in contrast, side select pin of the controller 32A in the left-footed configuration of the self-identification circuit 52B is instead connected to ground. In self-detection of its left or right footed status, the controller 32A thus simply checks whether the side-select input pin has a high or low voltage status, and assigns a right or left status according to whether that pin reads high (e.g. 3.3V) or low (ground). It will be appreciated that the choice of whether left or right footed status correlates to high or low voltage status is arbitrary, and not limited to the illustrated choice of such high/low-right/left implementation scheme.

The purpose of such self-identification is to eliminate or reduce the possibility of user-error during wireless pairing of the two insoles of a pair of footwear to the smartphone or other external receiving device that is to receive the reporting signals from the two insoles, to ensure that the measurement data in the reporting signals from each insole is properly displayed and/or logged by the smartphone software in properly mapped relation to the particular foot from which that data was collected. The controller 32A, once having identified its left-footed or right-footed status by checking its connected self-identification circuit 52A, 52B, thus assigns an appropriate “LEFT” or “RIGHT” identifier as part of a unique device identifier of the insole, which unique device identifier is then wirelessly broadcast by the transmitter in advertisement of the smart insole 10 as a wireless device pairable to the smartphone or other external receiving device. The unique device identifier may be, for example, a combination of this assigned “LEFT” or “RIGHT” identifier, and a MAC address of the Bluetooth controller that controls the Bluetooth transmitter.

FIG. 10 shows a screenshot of a setup screen in one non-limiting example of a graphical user interface (GUI) of the software application running on the smartphone or other external receiving device. This setup screen has a selectable “connect insoles” option 56 by which the user initiates initial pairing of left and right smart insoles of the present invention. In response to selection of the “connect insoles” option, the GUI invites sequential and individual pairing of the left and right insoles, so that during the pairing process for each, the user knows whether to choose a wirelessly pairable smart insole whose advertised device identifier includes a “LEFT” identifier, or one whose advertised device identifier includes a “RIGHT” identifier. After such pairing of both insoles is completed, the setup screen may display the paired device identifiers in respective association with right and left connection-status indicators 58A, 58B, which serves as visual confirmation to the user that the connected insoles are properly mapped as left and right inputs to the local software of the smartphone. With reference to FIG. 9, a sensor data display screen of the GUI is shown therein as being equipped with a left-right toggle option 60 by which the user can switch back and forth between display of measurement data from the left and right insoles, with confidence that the left and right data sets are properly mapped to the display based on the guided pairing process using the left and right specific device identifiers of the left and right insoles.

The tasks ascribed herein to the controller 32A of each smart insole 10 may be executed by embedded firmware thereof. At a high level, the embedded firmware of preferred embodiments performs a repeating loop, in which it reads measurement data from the sensors (e.g. temperature/force and ammonia), stores that measurement data in flash memory, sends any stored measurement data to the smartphone application if connected, and then enters a sleep cycle before repeating the loop. This loop contains four phases – startup, acquiring and storing the sensor data, transmitting reporting signals to the smartphone application to communicate the stored data thereto, and sleep. When the smart insole is first powered up, before initiating the looped routine, the firmware performs a few initialization tasks, including activating the Bluetooth controller and assigning the unique device identifier in the manner described above to include a left or right identifier therein, and standardizing the temperature sensor outputs in embodiments equipped with temperature sensors. In such embodiments, the temperature sensor outputs may be brought into agreement by applying an offset, where necessary, to accommodate any variation in value. These offsets are applied to all future temperature sensor readings and are recalculated on power loss. Once the initialization is complete, the controller proceeds to the second phase – reading the sensors.

During the sensor reading phase, the embedded firmware records sensor readings from the force/temperature sensors, and also from the optical sensor setup 24, and attaches a timestamp from a real time clock (RTC) to this measurement data. The firmware may apply software filtering to smooth out any minor variances in the sensor readings using filters present in the hardware and simple software averaging. After compiling a complete sensor data package, the firmware stores this data in flash memory. With a sensor data package in flash memory, the firmware checks for an active connection to the smartphone application. If there is no connection, the firmware skips this step. However, if the smartphone application connection is detected, the firmware sends single data packets to the software application of the smartphone and awaits acknowledgement after each transmission. If the firmware fails to receive an acknowledgement or runs out of data packages to send, the firmware exits this transmission phase and proceeds to the final phase – sleep.

The final sleep phase involves placing as many components as possible into an idle or sleep state, including the main microcontroller, though the Bluetooth controller will typically remain powered and active to ensure the firmware can respond to request signals from the smartphone application in real time, without waiting for expiration of the sleep phase.

This algorithmic implementation of the data acquisition, data recordal, data transmission and sleep steps in repeatedly looped fashion is just one non-limiting example for implementing the generally described and claimed functionalities of the smart insole, other workable alternatives of which may instead be adopted without escaping the bounds of the claimed invention set forth below.

While the forgoing embodiments are focussed on use of the optical sensor and colour-change membrane for detection of ammonia in the foot sweat of a user, other substance responsive colour-changing sensing membranes for detecting or measuring other substances present in sweat or other secreted or extracted bodily fluids of a user may be implemented in similar fashion for comparable purpose of detecting or measuring health-relevant substances found within one or more bodily fluids of the user exposable to such sensing membranes. It will also be appreciated that the optical sensing and colour-changing membrane combination useful for such purpose can be employed in various wearable smart devices, which need not necessarily be limited specifically to footwear articles or insoles for same, and may include other wearable smart devices, (gloves/mitts, wristbands, wristwatches, armbands, leg bands, neckbands, hats, skin attachable patches, etc.) for use anywhere a wearable body of the device is worn in close (though not necessarily immediate) adjacency to the skin of a user’s body part (arm, leg, hand, foot, torso, neck, head, etc.) for exposure to sweat or other excretions or extracted body fluids that may be useful to detect or measure a biometric or other health indicator gaugeable from such bodily fluid.

Likewise, it will be appreciated that the self-identifying “LEFT” and “RIGHT” designations of such wearable smart devices need not necessarily be limited specifically to left-footed and right-footed footwear articles or insoles for same, and may be exploited in any variety of wearable smart devices that are likewise sold and used in pairs wearable on any two bilateral appendages, whether such devices are worn on the left and right feet, calves, knees, thighs or other part of the user’s left and right legs, or on the left and right hands, wrists, forearms, biceps, shoulders, or other part of the user’s left and right arms.

Since various modifications can be made in the invention as herein above described, and many apparently widely different embodiments of same made, it is intended that all matter contained in the accompanying specification shall be interpreted as illustrative only and not in a limiting sense.

1. A wearable smart device with sensing capability, said wearable smart device comprising:
a wearable body wearable in adjacency to a body part of a user; and
in physical accompaniment to said wearable body:
a colour-changing sensing membrane positioned at a location exposable to a substance of interest during worn use of the wearable smart device by said user;
an optical sensor positioned to encompass at least a portion of the colour-changing sensing membrane within a field of view of said optical sensor; and
a light source positioned and operable to illuminate at least said portion of the colour-changing sensing membrane; and
an electronic controller operatively coupled to said optical sensor and light source, and configured to trigger:
execution of measurements comprising:
activation of the light source to cause illumination of at least said portion of the colour-changing sensing membrane; and
during said illumination, obtaining an output signal from the optical sensor that comprises, at least, colour data representative of a current colour of the colour-changing sensing membrane.

2. The wearable smart device of claim 1 further comprising a transmitter, and wherein the controller is also configured to trigger, after one or more of said measurements, transmission, by the transmitter, of reporting signals signifying one or more results of said one or more measurements.

3. The wearable smart device of claim 1 wherein said colour-changing sensing membrane is an ammonia sensing membrane.

4. The wearable smart device of claim 1 wherein at least one of the sensing membrane, the optical sensor, and the light source is at least partially embedded in the wearable body.

5. The wearable smart device of claim 1 wherein the optical sensor and the light source are hosted in a recessed cavity of the wearable body.

6. The wearable smart device of claim 5 wherein the wearable body has a multi-layered construction, and the recessed cavity is recessed into a surface of one of two adjacently stacked layers of said multi-layered construction at an interface of said two adjacently stacked layers.

7. The wearable smart device of claim 5 wherein the sensing membrane is hosted outside said recessed cavity in gap-spaced, but exposed, relation to the light source and the optical sensor through a communicative opening that communicates the recessed cavity with a location of the sensing membrane.

8. The wearable smart device of claim 1 wherein the colour-changing sensing membrane resides in gap-spaced relationship to the light source and the optical sensor.

9. The wearable smart device of claim 1 wherein the sensing membrane resides is hosted at an exterior of the wearable body.

1010 The wearable smart device of claim 1 wherein the light source comprises a white LED.

11. The wearable smart device of claim 1 wherein said wearable smart device is a footwear article or insole.

12. A sensing method comprising:
hosting, by a wearable smart device, a sensing setup comprising:
a colour-changing sensing membrane positioned at a location exposable to a substance of interest during worn use of the wearable smart device;
an optical sensor positioned to encompass at least a portion of the colour-changing sensing membrane within a field of view of said optical sensor;
a light source positioned and operable to illuminate at least said portion of the colour-changing sensing membrane;
an electronic controller operatively coupled to said optical sensor and light source; and
by operation of said electronic controller, executing measurements comprising:
activation of the light source to cause illumination of at least said portion of the colour-changing sensing membrane; and
during said illumination, obtaining an output signal from the optical sensor that comprises, at least, colour data representative of a colour currently possessed by the colour-changing sensing membrane.

13. A wearable smart device with sensing capability, said wearable smart device comprising:
a wearable body wearable on one of two bilateral appendages of a user;
in physical accompaniment to said wearable body, at least one sensor operable to take measurements of one or more measurable conditions during worn use of the wearable smart device by said user;
an electronic controller operatively coupled to said at least one sensor to take said measurements therefrom; and
a transmitter connected to said electronic controller and operable to transmit reporting signals, to an external device, based at least partially on said measurements taken from the at least one sensor;
wherein said electronic controller is configured to self-detect whether the wearable smart device is adapted for a left or right one of the bilateral appendages based on an automated reading, by the electronic controller, of a left/right self-identification circuit coupled to said electronic controller.

14. The wearable smart device of claim 13 wherein said left-right self-identification circuit is coupled solely to a singular input pin of said electronic controller, and said automated reading by the electronic controller comprises checking whether said singular input pin occupies a high or low voltage status, each of which represents a respective one of either a left or right appendage designation of the wearable smart device.

15. The wearable smart device of claim 14 wherein said wearable smart device is a first wearable smart device accompanied by a second wearable smart device in cooperative formation of a pair of wearable smart devices, in which one of said first and second wearable smart devices is left-appendaged and the other of said first and second wearable smart devices is right-appendaged, and among which the singular input pin of the controller of the first wearable smart device is connected to an operating voltage and the singular input pin of the controller of the second wearable smart device is connected to ground.

16. The wearable smart device of claim 13 wherein the electronic controller is configured to assign a left or right identifier as part of a self-assigned identifier of the wearable smart device based on a self-read status of the left/right self-identification circuit.

17. The wearable smart device of claim 16 wherein said self-assigned identifier is a wirelessly advertised identifier by which the wearable smart device identifies itself to the external receiving device for the purpose of pairing therewith.

18. The wearable smart device of claim 13 wherein said transmitter is a Bluetooth transmitter.

19. A method of setting up the wearable smart device of claim 15 comprising:
(a) by operation of the respective controllers of the first and second wearable smart devices, self-identifying said first and second wearable smart devices as left appendaged and right appendaged by automated reading of the left/right self-identification circuits of the first and second wearable smart devices; and
(b) in electronically pairing respective transmitters said first and second wearable smart devices to the external device, using a self-identified left-appendaged status of one of said first and second wearable smart devices to map the reporting signals therefrom as left-appendage readings and likewise using a self-identified right-appendaged status of another of said first and second wearable smart devices to map the reporting signals therefrom as right-appendage readings.

20. The method of claim 19 wherein using said self-identified left-appendaged and right-appendaged statuses in step (b) comprises selecting, during a respective pairing of each of the first and second wearable smart devices to the external device, a self-assigned identifier of each wearable smart device that was self-assigned by the respective controller of that wearable smart device, and by which that wearable smart device wirelessly advertises itself to the external device for pairing purposes, which self-assigned identifier of each wearable smart device includes a self-assigned left or right identifier derived from said automated reading.
